(19) United States
(12) Patent Application Publication
(10) Pub. No.: US 2017/0042541 A1
(43) Pub. Date: Feb. 16, 2017

(54) METHODS AND APPARATUS FOR INHIBITING SCAR FORMATION

(71) Applicant: Zipline Medical, Inc., Campbell, CA (US)

(72) Inventors: Amir BELSON, Los Altos, CA (US); Alan SCHAER, San Jose, CA (US); Eric STORNE, Menlo Park, CA (US)

(21) Appl. No.: 15/337,768

(22) Filed: Oct. 28, 2016

Related U.S. Application Data
(63) Continuation of application No. PCT/US2015/028066, filed on Apr. 28, 2015.

(51) Int. Cl. A61B 17/08; A61F 13/02; A61B 17/00

(52) U.S. Cl. CPC A61B 17/085; A61B 17/00491; A61F 13/0246

(57) ABSTRACT

Apparatus for closing a surgical incision include a base having left and right panels, a force distribution structure coupled to each panel, and a closure component which releasably attaches to the force distribution structure to draw the inner edges of the panels together in order to close adhered tissue edges. The force distribution structures allow the inner edges of the panels to expand while restraining expansion of the outer edges of the panels and limiting elongation of the lateral dimension of the panels. The incision closure apparatus may be placed on skin or other tissue prior to forming the incision to be available to close said incision at the end of the surgical procedure. The incision closure apparatus can provide a template for application of medical adhesive to the tissue after closure.

### CROSS-REFERENCE

This application claims the benefit of U.S. Provisional Application No. 61/985,572, filed Apr. 29, 2014, which application is incorporated herein by reference.

The subject matter of this application is related to the following commonly owned patent applications: U.S. patent applications Ser. No. 13/685,909 [Attorney Docket No. 35383-705.301], filed Nov. 27, 2012; Ser. No. 13/414,176 [Attorney Docket No. 35383-706.301], filed Mar. 7, 2012; Ser. No. 13/096,602, now U.S. Pat. No. 8,439,945 [Attorney Docket No. 35383-707.201], filed Apr. 28, 2011; Ser. No. 13/286,378, now U.S. Pat. No. 8,313,508 [Attorney Docket No. 35383-707.301], filed Nov. 1,2011; Ser. No. 13/874,046 [Attorney Docket No. 35383-707.302], filed Apr. 30, 2013; Ser. No. 13/286,757, now U.S. Pat. No. 8,323,313 [Attorney Docket No. 35383-709.201], filed Nov. 1,2011; Ser. No. 13/665,160 [Attorney Docket No. 35383-709.501], filed Oct. 31, 2012; Ser. No. 14/180,564 [Attorney Docket No. 35383-709.502], filed Feb. 14, 2014; Ser. No. 14/180,524 [Attorney Docket No. 35383-712.201]1, filed Feb. 14, 2014; 61/964,477 [Attorney Docket No. 35383-713.101], filed Jan. 5,2014; and, 61/980,776 [Attorney Docket No. 35383-715.101], filed Apr. 17, 2014, the full disclosures of which are incorporated herein by reference.

### BACKGROUND

The present disclosure relates generally to medical apparatus and methods. More particularly, the present disclosure relates to apparatus and methods for forming and closing surgical incisions.

Surgical closure devices including an adhesive based patch with right and left panels are known. Of particular interest of the present disclosure, such devices are described in co-pending, commonly owned PCT application US 2010/000430, the full disclosure of which is incorporated herein by reference. As described in the PCT application, an adhesive patch is placed over a patient's skin at a site where it is desired to form a surgical incision. After the patch is placed, an incision is formed along an axial line extending through the middle of the patch. After it is formed, the incision can be opened to perform a desired procedure, and after the procedure is completed the incision may be closed by drawing the inner edges of the panels together with a clip, zipper, or other closure member.

The principal objective of such surgical closure devices is to improve healing and reduce scaring from the incision. This objective, however, has been inhibited by certain characteristics of the presently available devices. For example, the tissue edges are not always brought together along an even line, which can increase the eventual scaring. Many such closure devices do not have the ability to adjust the closure force or distance on the tissue edges, limiting the ability to slightly “pucker” tissue which has been found to reduce scaring. Other shortcomings of the available incision and wound closure devices include difficulty of use and inability to conform to tissue manipulation during subsequent surgical protocols, i.e. those devices which are sufficiently rigid to securely close the tissue are often unable to conform to the tissue movement during the surgical procedure.

For these reasons, it would be desirable to provide improved surgical incision closure devices and methods for their use. It would be particularly desirable to provide incision closure devices which are able to adhere to the tissue, allow formation of the incision, conform to the deformation of the tissue during a subsequent surgical procedure, provide controlled closure of the adjacent tissue edges subsequent to the procedure, and accelerate healing while reducing scarring. At least some of these objectives will be met by the systems, devices, and methods described below.

### SUMMARY

The present disclosure provides improved apparatus and methods for closing wounds, particularly wounds resulting from incisions performed during surgical procedures. The incisions would usually be formed in a patient's skin, such as through the abdomen, but in some cases could also be on internal organs, within the oral cavity, within body cavities, or alike.

The devices and methods of the present disclosure will present minimum disruption of or interference with the surgical procedure which is performed after the incision is made. In particular, the devices and methods will permit the opposed edges of the incised tissue to be opened, stretched, and freely deformed with minimal restraint resulting from the presence of the closure device. Once the procedure has been completed, however, the devices and methods of the present disclosure will provide for a uniform distribution of closure forces to draw the tissue edges together in a manner which and minimize scaring. In particular, the closure devices can draw the tissue edges together at a slightly closer spacing than initially present at the forming of the incision in order to upwardly evert the tissue edges cause a “pucker” which can reduce scaring. Moreover, the devices and methods will permit the opposed edges of the incised tissue to be aligned as they were prior to incision and thereby providing a template to facilitate the application of medical adhesives to the incised tissue. Devices and methods for application of medical adhesives are further provided herein.

An aspect of the present disclosure provides a method of forming an incision in tissue. Right and left base panels of an incision closure apparatus may be adhered to a surface of the tissue. The incision may be formed in the tissue surface between inner edges of the panels. The incised tissue edges may be separated to perform a procedure. The inner edges of the panels can stretch and conform along with the tissue edges while an outer edge and lateral extent of each panel remain dimensionally stable. A plurality of closure components may be secured to the right and left base panels to draw the inner edges of the panels back together. An adhesive may be applied between the inner edges of the panels between axially adjacent closure components.

The adhesive may be applied with an external applicator. The adhesive may be applied by introducing an adhesive applicator between the inner edges of the panels. The adhesive applicator, when introduced between the inner edges of the panels, may be disposed over the tissue surface and optionally below the plurality of closure components. An absorbent liner of the adhesive applicator may further be used to absorb fluid from the incision. The absorbent liner may be peeled away from an adhesive layer of the adhesive applicator. The adhesive layer may be adhered to the tissue surface and a stiff member of the adhesive applicator may be removed from the adhesive layer. The adhesive may comprise one or more of a cyanoacrylate, fibrin, albuim, or a glutaraldehyde based adhesive.

Another aspect of the present disclosure provides an apparatus for applying an adhesive to a surface. The apparatus may comprise an elongate stiff member, an adhesive layer, and an absorbent liner. The elongate stiff member may have a bottom surface. The adhesive layer may be removably coupled to the bottom surface of the elongate stiff member. The adhesive layer may have an adhesive bottom surface. The absorbent liner may be removably coupled to the adhesive bottom surface of the adhesive layer. The elongate stiff member may have a distal end and the adhesive layer may comprise a distal tab disposed distally of the distal end. The elongate stiff member may have a proximal end and the absorbent liner may comprise a proximal tab disposed proximally of the proximal end. The adhesive layer may comprise one or more of a cyanoacrylate, fibrin, albuim, or a glutaraldehyde based adhesive. The absorbent liner may be folded onto itself

Another aspect of the present disclosure provides a kit for forming an incision in tissue. The kit may comprise an incision closure apparatus comprising a base, a force distribution structure, and a closure component. The base may include a left panel and a right panel. Each panel may have a tissue adherent lower surface, an upper surface, an inner edge, and an outer edge. The force distribution structure may be coupled to each panel. Each force distribution structure may include a spine disposed axially adjacent the inner or outer edge to limit axial expansion along the edge and a plurality of axially spaced-apart structures extending laterally from the spine toward the inner or outer edge to limit lateral expansion while allowing axial expansion along the edge. The closure component may releaseably attach to the force distribution structure to draw the inner edges of the panels together after they are adhered to tissue on opposite sides of an incision. The kit may further comprise an adhesive for application to the tissue. The kit may further comprise instructions to apply the adhesive to tissue between the inner edges of the panels after the panels are adhered to tissue on the opposite sides of the incision.

The adhesive may comprise one or more of a cyanoacrylate, fibrin, albuim, or a glutaraldehyde based adhesive. The kit may further comprise an adhesive applicator for applying the adhesive. The adhesive applicator may comprise one or more of a spray gun, an applicator tube, an applicator pen, or a brush. For example, the adhesive applicator may comprise an elongate stiff member having a bottom surface, an adhesive layer removably coupled to the bottom surface of the elongate stiff member and having an adhesive bottom surface, and an absorbent liner removably coupled to the adhesive bottom surface of the adhesive layer.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 is an exploded view of an incision closure appliance constructed in accordance with the principles of the present disclosure.

FIG. 2 is a top view of the assembly of a base and a force distribution structure which is part of the incision closure appliance.

FIG. 3 is a cross-sectional view taken along line 3-3 of FIG. 2.

FIGS. 4-7 illustrate use of the incision closure appliance of the present disclosure for forming and closing an incision in a patient's skin.

FIG. 8 illustrates an alternative construction of a closure component for the closure appliance of the present disclosure.

FIG. 9 is an exploded view of a further embodiment of an incision closure appliance constructed in accordance with principles of the present disclosure.

FIG. 10 is an enlarged isometric view of the base and force distribution structure of the system of FIG. 9.

FIGS. 11A and 11B illustrate an alternative lateral tie construction which can be used in the appliances of either FIG. 1 or FIG. 9.

FIG. 12 illustrates a sacrificial cover positioned over an incision closure appliance in accordance with the principles of the present disclosure.

FIGS. 13A through 13E illustrate the principle of operation of the sacrificial cover illustrated in FIG. 12 when sued together with a surgical incision drape and performing methods according to the present disclosure.

FIG. 14 illustrates the closure component of FIG. 8 and open areas provided by the closure component for the application of medical adhesive.

FIG. 15 illustrates the incision closure appliance of FIGS. 9 and 10 and open areas provided by the incision closure appliance for the application of medical adhesive.

FIG. 16A shows a perspective view of a medical adhesive applicator constructed in accordance with principles of the present disclosure.

FIG. 16B shows a cross-sectional view of the medical adhesive applicator of FIG. 16A.

FIGS. 17A through 17E show the medical adhesive applicator of FIG. 16A used to apply medical adhesive over an incision in tissue.

FIGS. 18A through 18D show further medical adhesive applicators constructed in accordance with principles of the present disclosure.

### DETAILED DESCRIPTION

The apparatus and methods of the present disclosure will be used during both the formation and the closure of surgical incisions made to a patient's skin or other tissue during surgical procedures. As described hereinafter, the direction of the incision will define both “axial” and “lateral” directions as those terms are used herein. Most incisions will be made along a generally straight line which will define the axial direction. The lateral direction will generally be across the axial direction, typically but not necessarily being perpendicular or normal to the axial direction. Most incisions will be generally linear but in some cases the incisions could be curved or have other geometries. The term “axial” will then apply to the direction of the incision at any particular location, resulting in lateral directions which could also vary.

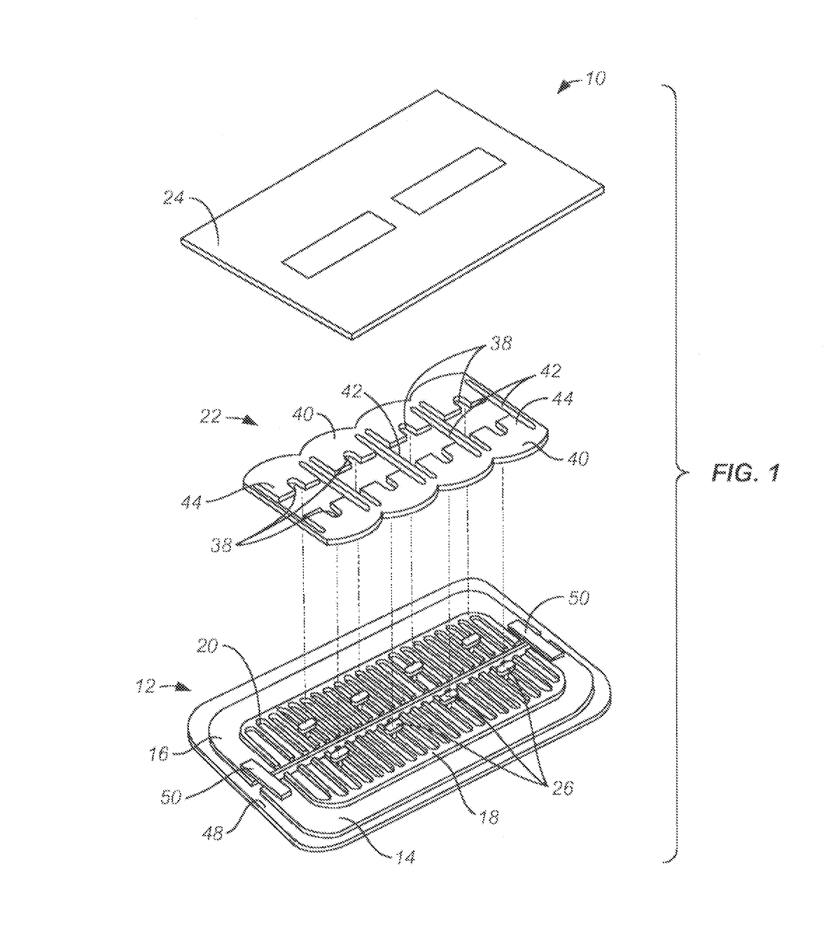

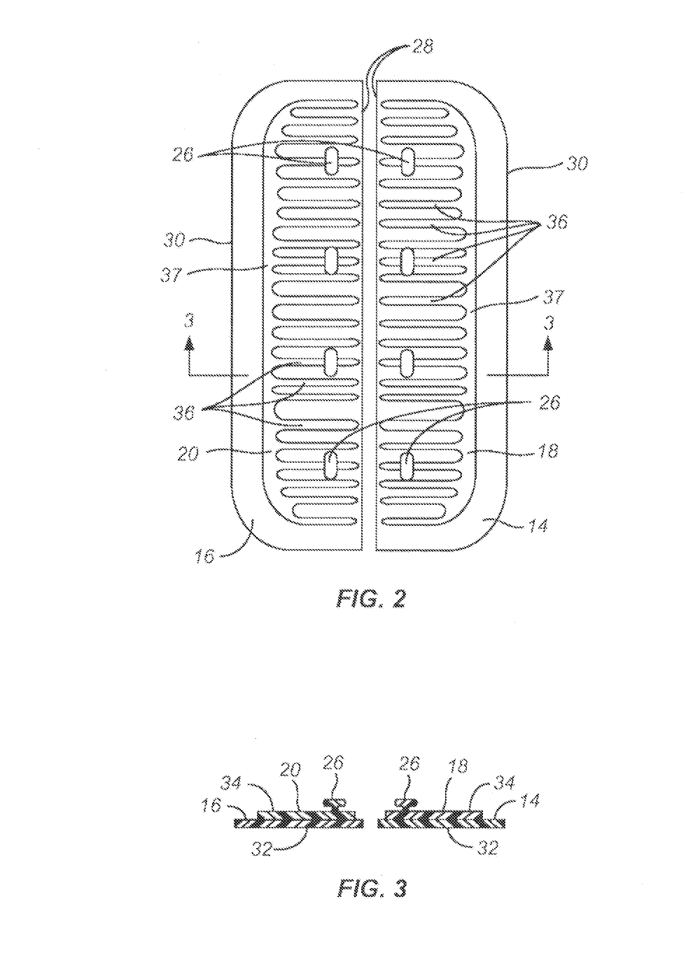

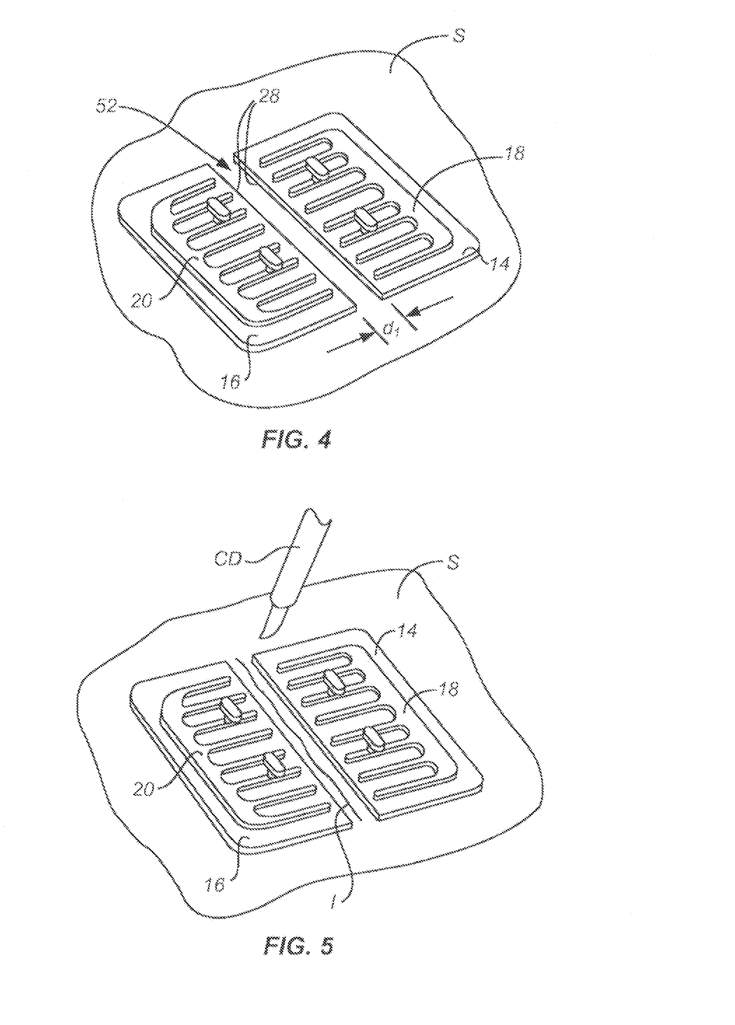

Referring now to FIGS. 1-3, an incision closure appliance 10 comprises a base assembly 12 including a right panel 14 and a left panel 16. A right force distribution structure 18 is secured to the right panel 14, typically by laminating the force distribution structure to an upper surface of the panel, and a left force distribution structure 20 is similarly attached to an upper surface of the left panel 16. The incision closure appliance further comprises a closure component 22 which is removably attachable to the right and left forced distribution structures 18 and 20 in order to close an incision, as described in more detail below, and the appliance is completed with an optional securing layer 24 which may be placed over the combined base assembly 12 and closure component 22 after they have been secured to the patient and the incision has been closed by drawing the panels together using the closure component.

The closure component 22 is intended and adapted to draw the inner portions of the force distribution structures 18 and 20 inwardly toward each other to close a surgical incision which has been formed therebetween. In the illustrated embodiment, a plurality of cleats 26 are formed on lateral supports 36 which are held axially by spine 37 of the force distribution structures 18 and 20. The cleats 26 are received in slots 38 formed along inner edges of opposed engagement members 40 of the closure component 22. The opposed engagement members 40 are held together by lateral struts 42 so that the engagement members are held at a fixed, laterally spaced-apart distance (in other embodiments the spaced-apart distance may be adjustable). The slots 38 are preferably formed on flexible tab-like structures 44 which allow the slots to be pulled upwardly over the corresponding cleats in order to secure the closure component 22 over the force distribution structures 18 and 20.

The lower surfaces 32 of each panel 18 and 20 will typically be covered with a pressure-responsive adhesive, where the adhesive is initially covered with a protective layer 48 which may be peeled away immediately prior to use. Additionally, pull-away tabs 50 or other similar structures may be provided in order to hold the right and left panels 14 and 16 together at a pre-determined spaced-apart distance after the layer 48 has been removed but prior to adhering the panels to a patient's skin or other tissue surface. It is important that the distance between the inner edges 28 of each panel 14 and 16 be maintained as close as possible to the original target spacing so that the tissue edges, when closed by the closure component 22, will be precisely brought together, typically with a slight eversion.

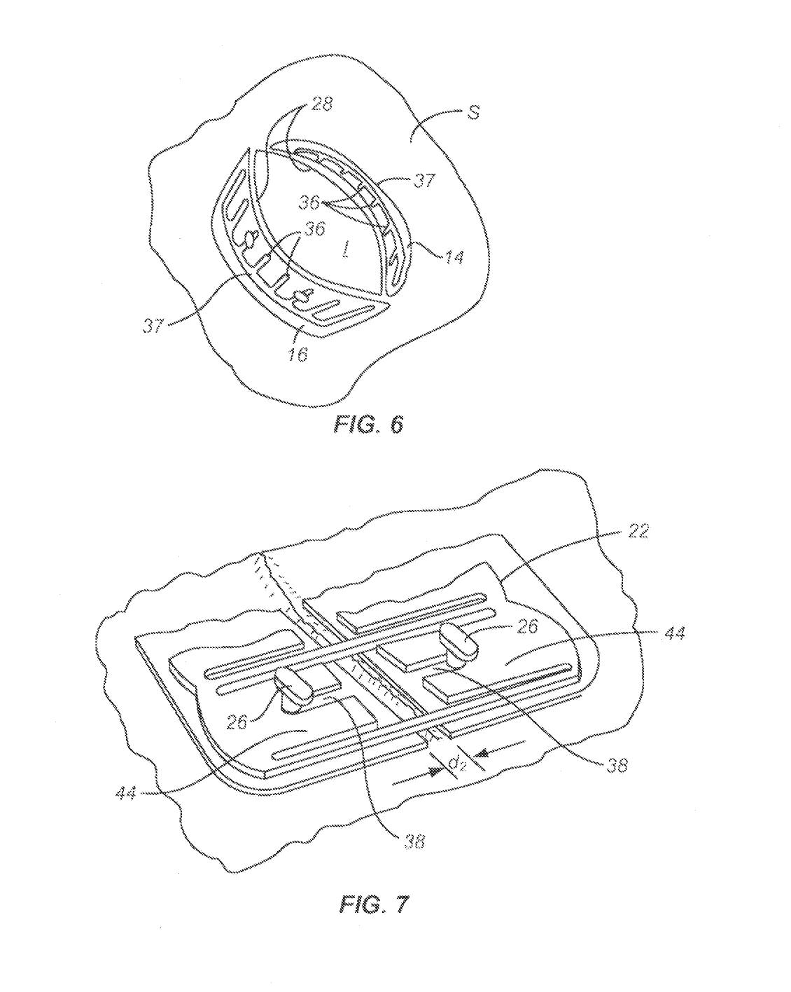

Referring now to FIGS. 4 through 7, a protocol for both forming an incision and subsequently closing the incision in accordance with the principles of the present invention will be described. Initially, the right and left panels 14 and 16 are placed on the patient's skin followed by reference letter S, as shown in FIG. 4. The panels 14 and 16 are applied by first pulling away the protective layer 18 and placing the panels onto the tissue, after which time the tabs 50 may be removed, leaving an incision path 52 defined between the inner edges 28. The spacing of the inner edges 28 will be selected to provide a fixed, pre-determined distance d1.

After the right and left panels 14 and 16 are in place, an incision I can be formed in the space between the panels using a scalpel or other surgical cutting device CD, as shown in FIG. 5.

After the incision I is made, a surgical procedure may be performed by opening the inner edges of the incision which in turn deforms the inner edges 28 of the right and left panels 14 and 16, as shown in FIG. 6. As the inner most ends of the supports 36 are not connected, they are free to separate and allow the elastic matrix of the right and left panels 14 and 16 to expand, as clearly in FIG. 6. The dimensional stability of the remainder of the panels, however, will be preserved by the lateral supports 36 as well as the axial spines 37 which do not elongate under the influence of the force applied by stretching opening the incision.

After the surgical procedure is complete, the closure component 22 will be secured over the force distribution structures 18 and 20, as illustrated in FIG. 7. In particular, the slots 38 in the tab-like structures 44 are engaged over opposed cleats 26 in order to draw opposed edges of the panels as well as of the tissue incision together. By properly spacing the depth of the slots 38, the closure component 22 can be tailored so that the panels 14 and 16 are brought together by a pre-selected distance d2. Typically, the distance d2 will be less than the initial separation d1 so that the inner edges of the tissue are brought together to cause the tissue edges along the incision to slightly evert (pucker upwardly) which can improve healing and reduce scarring.

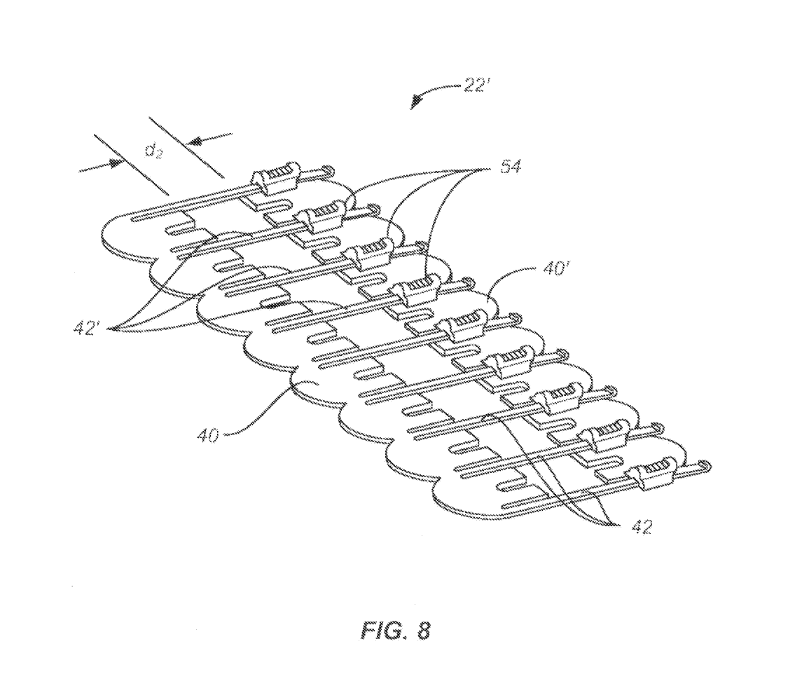

Optionally, as shown in FIG. 8, a closure component 22′ may include engagement members 40′, where one end of each lateral strut 42′ is joined by an adjustable clasp or other mechanism 54 so that the distance between the inner edges of the opposed engagement members 40′ can be adjusted in order to increase or lessen the distance d2 therebetween.

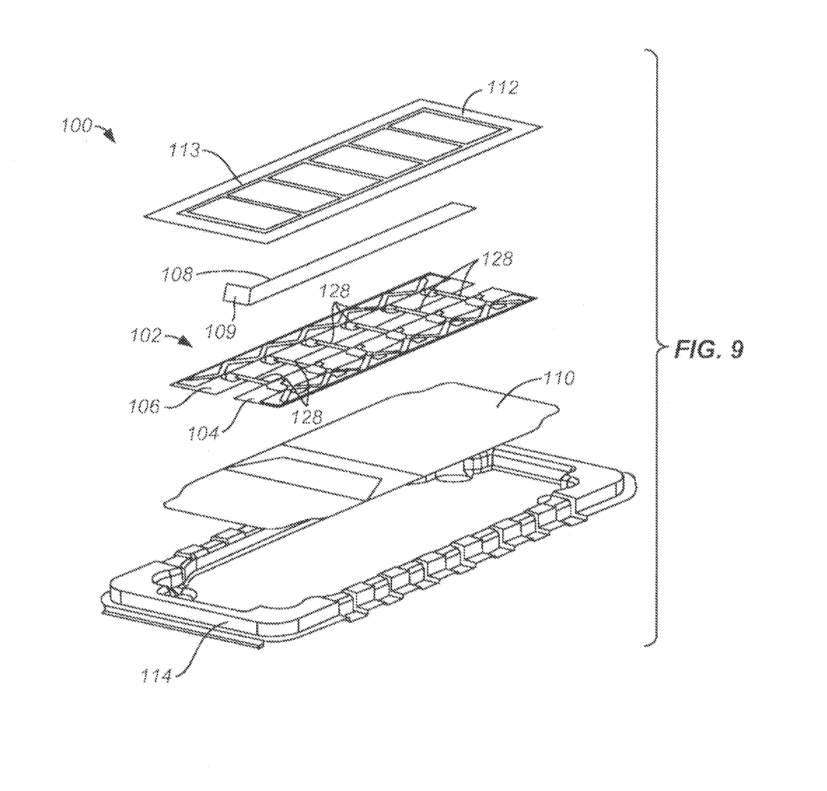

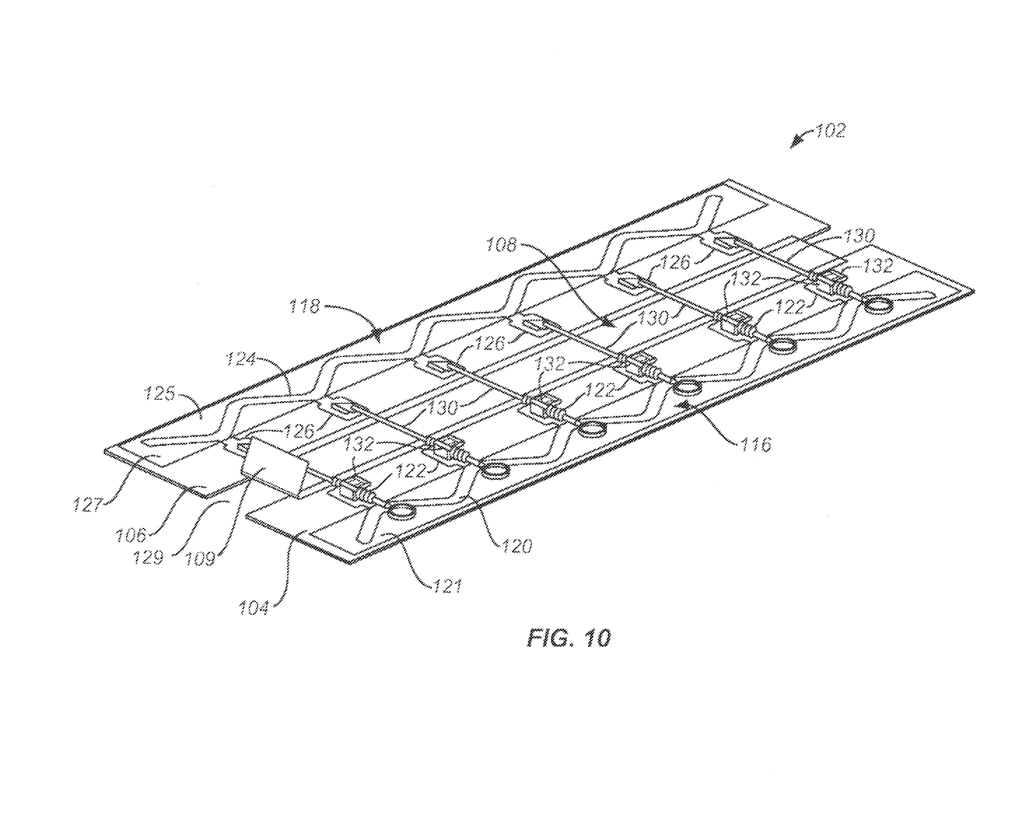

An alternative embodiment 100 of the incision closure appliance of the present invention is illustrated in FIGS. 9 and 10. The appliance 100 includes a base assembly 102 having a right panel 104 and a left panel 106. A positioning or alignment strip 108 is provided to secure the inner edges of each panel together, as shown best in FIG. 10 and includes an end tab 109 that allows the user to pull the strip from the panels 104 and 106 after the panels have been put in place on a tissue surface.

The incision closure appliance 100 further includes a backing 110 having an end which may be partially folded back to expose an underlying adhesive backing on the panels and allow that end of the base assembly 102 to be adhered to the tissue while the remainder of the base assembly is still covered by the backing. A securing layer 112 which includes a reinforcement frame 113 is provided for placement over the right panel 104 and left panel 106 after the base assembly 102 has been closed over an incision, generally is described in connection with the previous embodiment. Usually, a holding tray 114 will be provided for maintain the components of the appliance together in a sterilized condition where the tray 114 will be covered with conventional medical packaging cover.

As illustrated in FIGS. 9 and 10, a right force distribution structure 116 and a left force distribution structure 118 are provided on the upper surfaces of the right panel 104 and the left panel 106, respectively. The right force distribution structure 116 includes a right axial spine 120 and a plurality of lateral supports 122. Typically, the right axial spine 120 comprises a serpentine or zig-zag number which is embedded in or laminated to a base strip 121. The serpentine axial spine 120 would typically be formed from a flexible, resilient plastic, typically a hard plastic, while the base strip 121 will be comprised of a polyurethane or similar plastic layer. The lower surface of the polyurethane layer will be covered with a hydrocolloid layer for tissue adhesion. The structure of the left forced distribution structure 118 will be the same including a left axial spine 124, left lateral supports 126, and a left base strip 127.

The incision closure appliance 100 will include a closure mechanism comprising a plurality of lateral tie assemblies 128 as shown on FIG. 9. As best seen in FIG. 10, each lateral tie assembly 128 will include a rod which is secured at one end to the left lateral support 126 and a ratchet mechanism 132 which is secured to the right lateral support 122. Each rod 130 will usually be aligned with the axis of the left panel 106 parties so that a gap 129 between the right panel 104 and left panel 106 will be left open so that an incision can be made there between. After the incision is made, each rod 130 will be pulled over to the associated ratchet 132 on the right panel 104. A series of ratchet rings on each rod will be pulled into the associated ratchet mechanism 132, and the rod then pulled laterally until the desired closing tension is applied at that point along the base assembly 102. It is a particular advantage that each of the lateral tie assemblies 128 may be individually adjusted to supply the desired closing tension across the tissue along the length of the incision being closed. Once the desired closing tension has been provided along the entire incision, the securing layer 112 may be placed over base assembly 102 to hold the appliance and tissue in place.

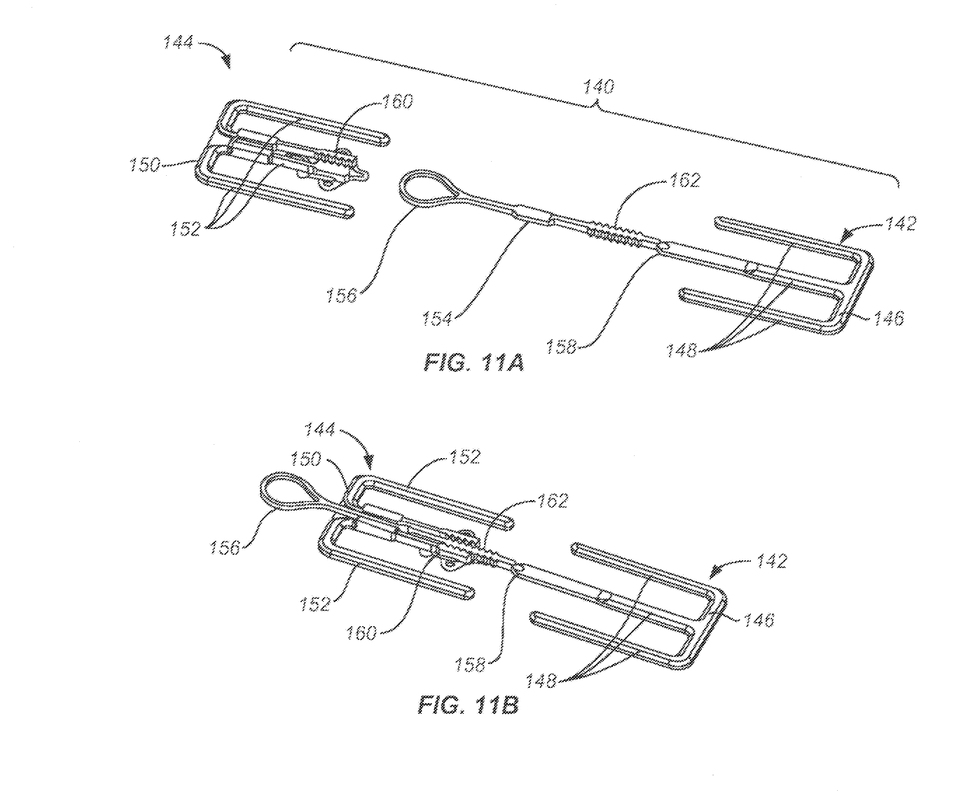

Referring now to FIGS. 11A and 11B, an alternative design for the lateral tie assemblies 140 of the present invention is illustrated. These lateral tie assemblies 140 may be utilized with either of the incision closure appliances 10 or 100 described previously. Each lateral tie assembly 140 includes a right force distribution structure 142 and a left force distribution structure 144. The right force distribution structure includes a right spine 146 and a plurality of lateral supports 148. Although three are shown, it will be appreciated that four, five, six or more lateral supports could be included. The left force distribution structure 144 similarly includes a left spine 150 and a plurality of left lateral supports 152. To provide closure, the right force distribution structure 142 includes a rod 154 which extends from the center lateral support 148. Typically, the rod 154 is joined to the support by a live or passive joint 158. A pull loop 156 is provided at the free end of the rod 154, and a plurality of ratchet teeth 162 are provided along the midsection of the rod 154.

The left force distribution structure 144 includes a ratchet mechanism 160 adapted to receive the teeth 162 on the rod 154 of the right force distribution structure. In this way, the rod 154 can be lowered into the ratchet 160 to engage teeth 162, allowing the rod to be pushed forward in order to draw the right and left force distribution structures 142 and 144 together in order to apply tension to the right and left panels.

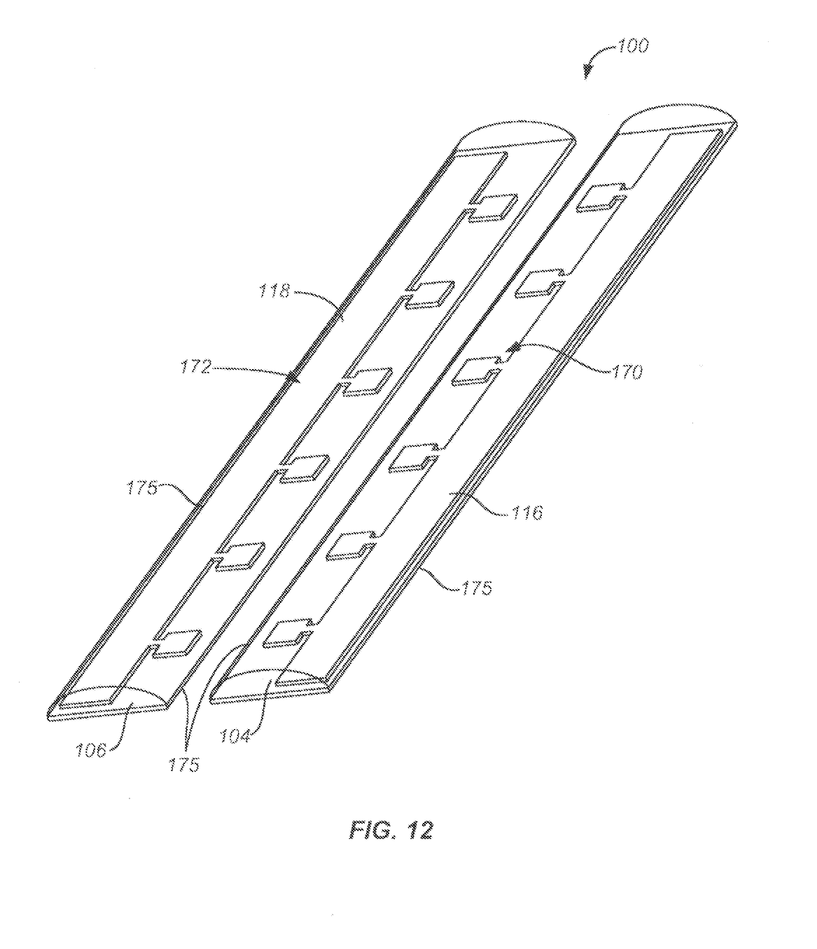

As illustrated in FIG. 12, a further aspect of the present disclosure is illustrated. The incision closure appliance 100 is illustrated schematically with only the right and left panels 104 and 106 and the right and left force distribution structures 116 and 118 being illustrated. The remaining system components are not shown for ease of illustration.

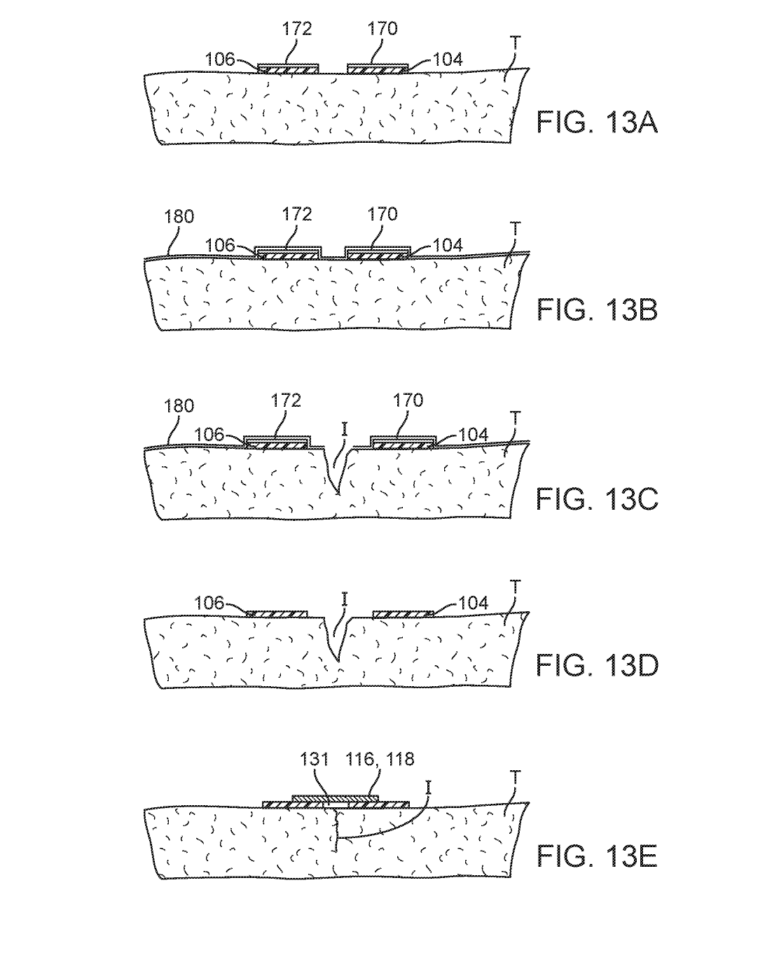

The right panel 104 is covered by a right sacrificial cover 170 and the left panel 106 is covered by a left sacrificial cover 172. The right and left panels 104 and 106 may define a region 175 where the right and left sacrificial covers 170 and 172 separates from the right and left panels 104 and 106. Each cover 170 and 172 is detachably secured along each edge of the associated base panel so that the covers remain in place during normal handling and placement of the incision closure appliance 100 over the tissue surface to be incised. The use and purpose of these sacrificial covers 170 and 172 is described with reference to FIGS. 13A and 13E.

FIG. 13A illustrates the right and left panels 104 and 106 in place on a tissue surface T prior to an incision being made. The right panel 104 is covered by right sacrificial cover 170 and the left panel 106 is covered by left sacrificial cover 172. As is common in many surgeries, an adherent surgical incision drape 180 is placed over the tissue surface T. Any conventional drape may be used such as the Ioban™ antimicrobial incise drape, available from 3M, St. Paul, Minn.

After the incision drape 180 is in place over the incision closure appliance, a surgical incision I may be made for performing a desired surgical intervention. As can be seen, the incision I will cut through the surgical drape 180 between the right and left panels 104 and 106, respectively. After the surgical procedure is completed, the surgical drape 180 will be removed from the tissue surface T. As the surgical drape has a lower adherent surface, prior to the present invention, removal of the drape might have displaced either or both of the right panel 104 and left panel 106. Presence of the sacrificial layers 170 and 172, however, prevents such displacement. Removal of the surgical drape 180 will remove the sacrificial layer 170 and 172, but as each of these layers is configured to break off with a relatively low separation force, removal of the sacrificial layers will not cause the underlying panels 104 or 106 to be displaced. Thus, the panels 104 and 106 will be left in place, as shown in FIG. 13D, and the force distribution structures 116 and 118 can be used as described previously for closing the panels together to close the incision as shown in FIG. 13E.

The incision closure appliances 10 and 100 may also be used for applying a medical or surgical adhesive (e.g. a cyanoacrylate, fibrin, albumin, or glutaraldehyde based adhesive) for closure of the skin incision I after surgery. The surgical adhesive may comprise a pressure-responsive adhesive. As discussed above, the components of the incision closure appliances 10 and 100 maintain the proper alignment of tissue after an incision has been made and subsequently closed. Applying the medical or surgical adhesive further maintains the proper tissue alignment.

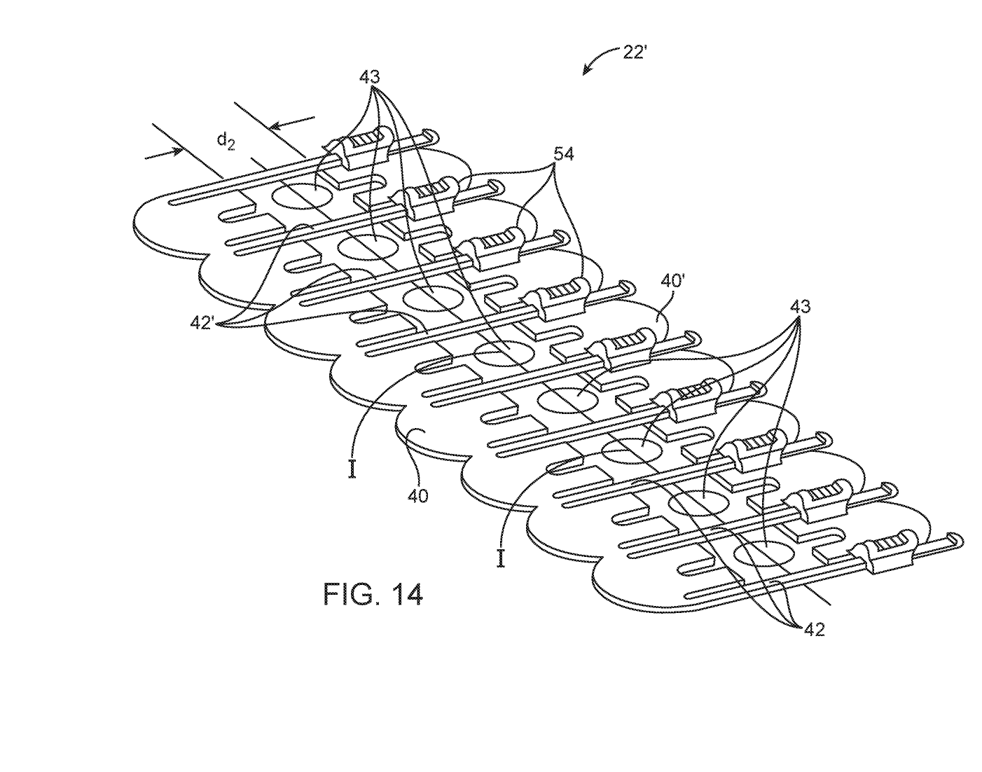

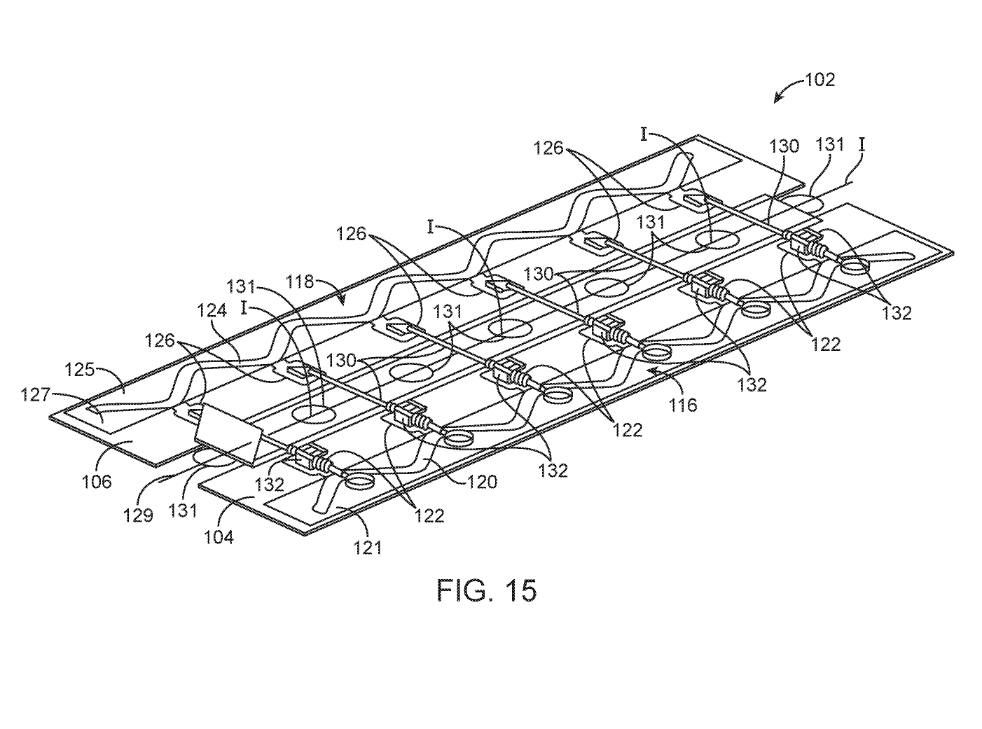

The incision closure appliances 10 and 100 can be used as a guide or template for the application of medical or surgical adhesives. At least a portion of the incision closure appliances 10 and 100 would be made of or coated with a material, such as PTFE or HDPE, which does not bond with the surgical adhesive. In using the incision closure appliance 10, the surgical adhesive may be applied using a conventional surgical adhesive applicator (e.g., a spray gun, an applicator tube, an applicator pen, a brush, etc.) onto adhesive application areas 43 axially between the lateral struts 42′ of the closure components 22 (see FIG. 14). The surgical adhesive will generally be applied after the incision I has been properly aligned and closed using the right panel 14, the left panel 16, and the closure component 22. In using the incision closure appliance 100, the surgical adhesive may be applied to the skin incision I after the force distribution structures 116 and 118 have been used to close the panels 104, 106 together and properly align the tissue T. The surgical adhesive may be applied using a conventional surgical adhesive applicator onto the adhesive application areas 131 axially between the rods 130 of the lateral tie assemblies 128 (see FIG. 15). FIG. 13E shows an adhesive application area 131 in cross-section as located below the force distribution structures 116, 118, between the panels 104 and 106, and above the incision I and tissue T. Once applied, the surgical adhesive may rapidly cure upon contact with the tissue T to hold the aligned incision I in place.

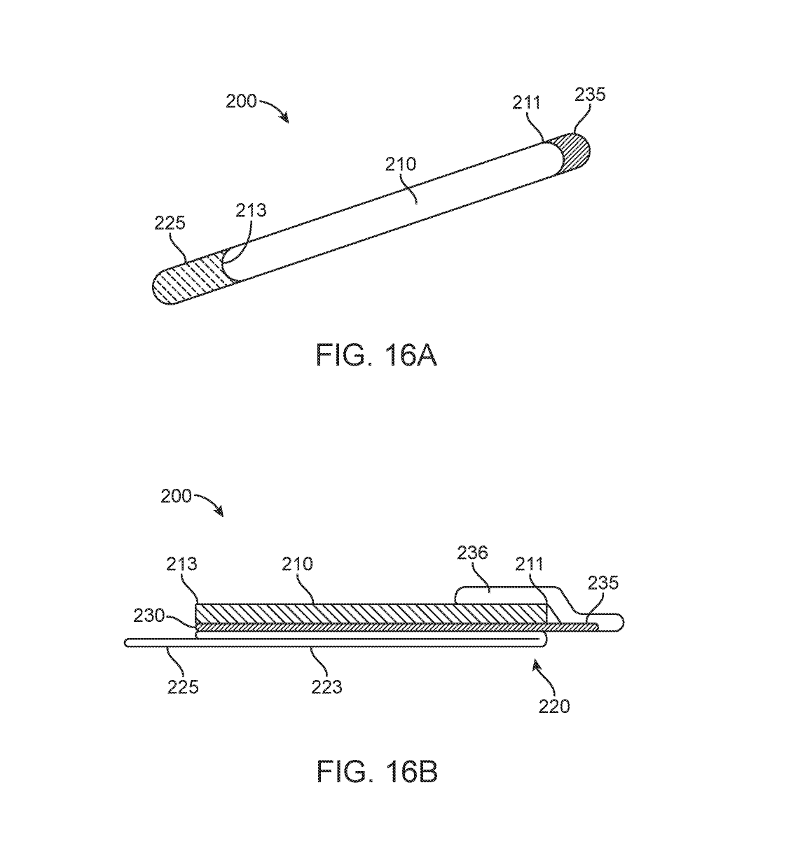

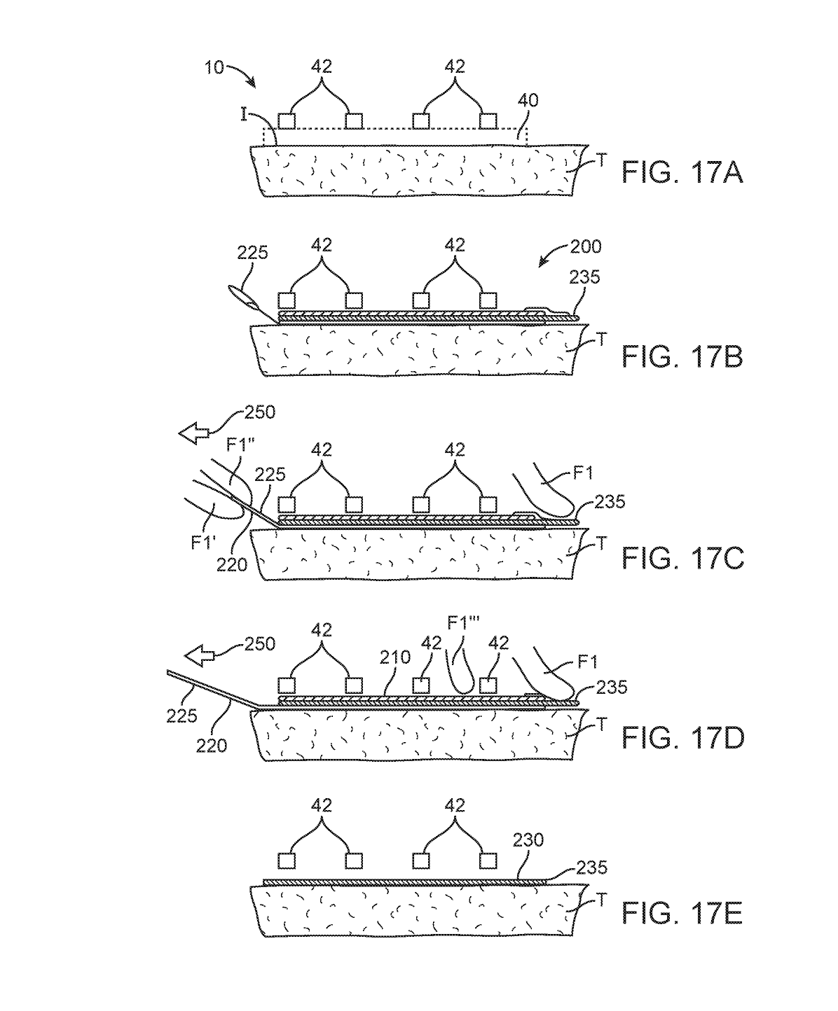

Alternatively or in combination, a medical adhesive applicator configured for use with the incision closure appliances 10 and/or 100 may be used to apply the medical or surgical adhesive (e.g., a cyanoacrylate, fibrin, albuim, or glutaraldehyde based adhesive) to close the skin incision I in tissue T after surgery. FIG. 16A shows a perspective view of a medical adhesive applicator 200, and FIG. 16B shows the medical adhesive applicator 200 in cross-section. The applicator 200 may comprise a stiff elongate member 210 having a distal end 211 and a proximal end 213. An adhesive layer 230 may be coupled to the bottom surface of the elongate member 210. The adhesive layer 230 may have an adhesive bottom surface and/or an adhesive upper surface. The adhesive layer 230 may provide for stronger adhesion at its bottom surface rather than its upper surface so that the elongate member 210 can be readily separated from the adhesive layer 230 when the adhesive layer 230 is adhered to the tissue T. Alternatively or in combination, the upper surface of the adhesive layer 230 may be coated with a thin, non-adhesive layer (e.g., polyurethane) and/or the bottom surface of the elongate member 230 may be removably coupled with the upper surface. Alternatively or in combination, the elongate member 210 may be fixedly attached to the adhesive layer 230. The adhesive layer 230 may comprise a distal tab 235 extending beyond the distal end 211 of the elongate member 210. The distal tab 235 may have a non-adhesive upper surface or may be provided with a non-adhesive cover 236. An absorbent liner 220 may be coupled to the bottom surface of the adhesive layer 230. The absorbent liner 220 is folded onto itself such that the liner 220 may be double-layered as shown in FIG. 16B. The absorbent liner 220 may comprise an absorbent bottom surface 223. The absorbent liner 220 may comprise a proximal tab 225 which can be used to pull back in the proximal direction to retract the inner 220 from the bottom surface of the adhesive layer 230. The absorbent liner 220 may be adhesion resistant such that the elongate member 210 remains adhered or fixed to the adhesive layer 230 as the absorbent liner 220 is peeled away from the adhesive layer 230.

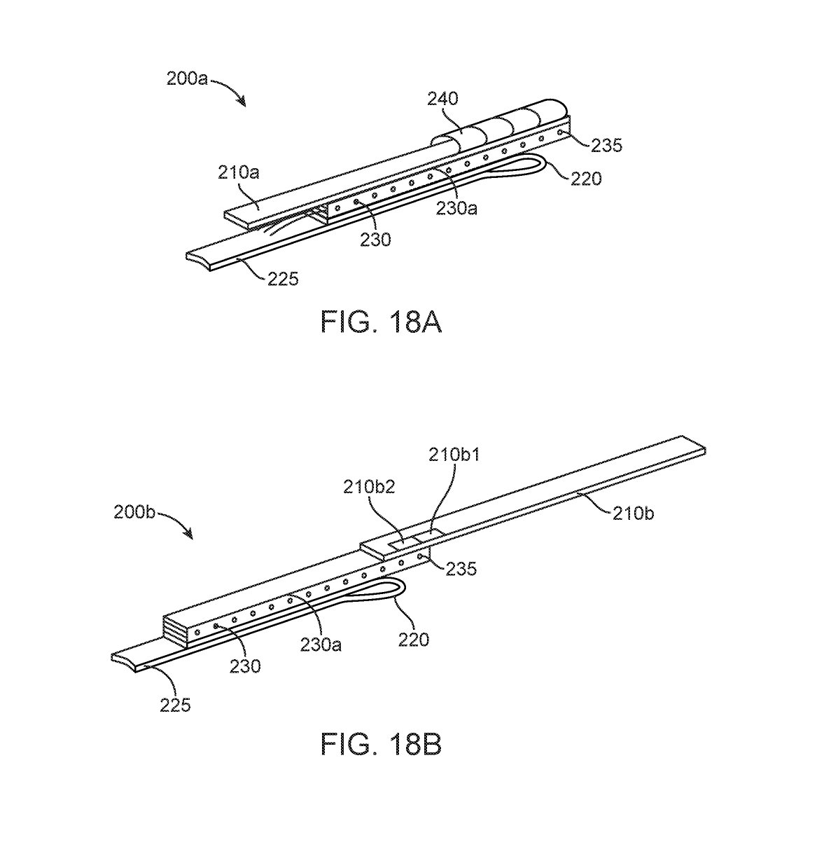

FIGS. 17A through 17E show the medical adhesive applicator 200 used to apply medical adhesive over an incision I in tissue T. FIG. 17A shows the incision closure appliance 10 positioned over tissue T. Generally, the incision closure appliance 10 has been used to properly align and close the incision I. As shown in FIG. 17A, there is some space 40 between the upper surface of the tissue T and the bottom surfaces of the lateral struts 42. FIG. 17B shows the medical adhesive applicator 200 being inserted axially into this space 40 with the lateral struts 42 positioned over the adhesive applicator 200. Alternatively, the lateral struts 42 may be thin and low-profile (e.g., 0.005″-0.010″ thick and 1-4 mm wide) so that the medical adhesive applicator 200 is applied over the lateral struts 42. As shown in FIG. 17C, a user can apply pressure with his/her finger FI to distal tab 235 to stabilize the position of the tissue closure appliance 10 relative to the incision I and the tissue T. The user can also use his/her fingers FI′ and FI″ to grasp the proximal tab 225 of the absorbent liner 220 in preparation to peel back the liner 220 in the proximal direction as shown by arrow 250. As shown in FIG. 17D, the absorbent liner 220 can be gradually pulled back in the proximal direction as shown by arrow 250. As the absorbent liner 220 is gradually peeled back, the liner 220 absorbs any fluid such as blood oozing out of the incision I. Immediately after such absorption, the adhesive layer 220 can be adhered to the upper surface of the tissue T at the incision I. The adherence of the adhesive layer 220 can be facilitated with the application of pressure using another finger FI″ of the user. For example, the adhesive layer 230 may comprise a pressure-responsive adhesive. By first absorbing any extraneous fluid with the liner 220, the adhesive layer 230 can adhere to more of the surface of tissue T to provide stronger, more-lasting adhesion. In other words, there will often not be enough extraneous fluid to form a substantial fluid layer between the upper surface of the tissue T and the bottom layer of the adhesive layer 230 which would otherwise disrupt adhesion. As shown in FIG. 17E, after the absorbent liner 220 is completely peeled away, the stiff elongate member 210 may be separated from the adhesive layer 230 and removed to leave only the adhesive layer 230 adhered to the upper surface of tissue T. The top layer of the adhesive layer 230 may comprise a weak adhesive or may comprise an adhesive surface which may have its adhesive properties neutralized to prevent unwanted adhesion to other dressing, clothing, etc. Alternatively, the stiff elongate member 210 may be left adhered to the top layer of the adhesive layer 230. Or, the stiff elongate member 210 may be removed and replaced with a more flexible liner.

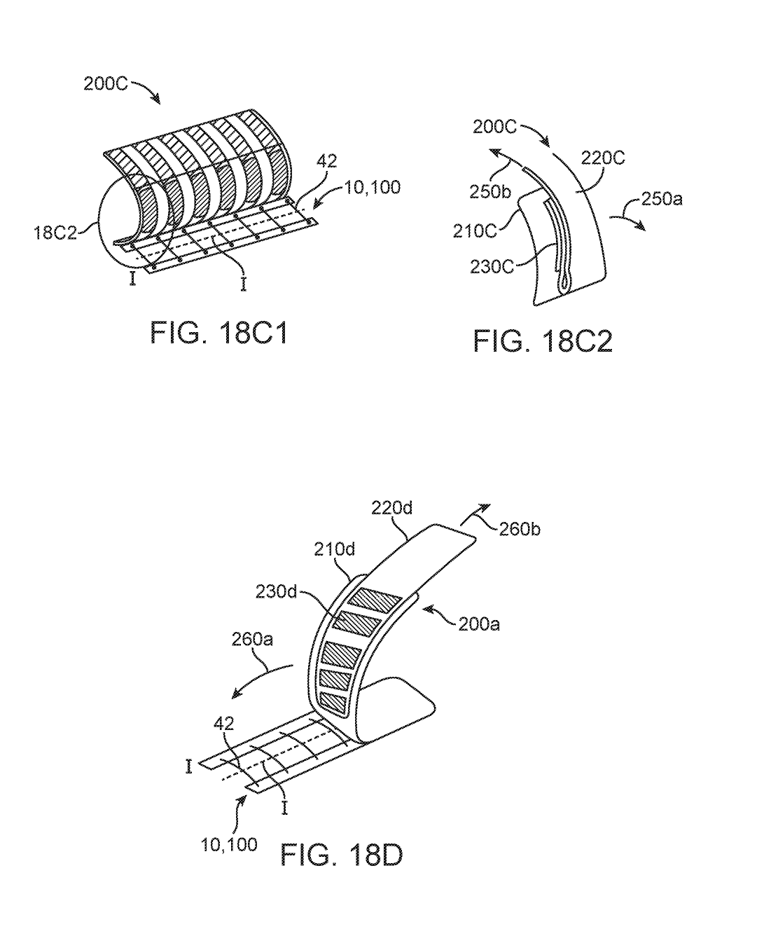

FIGS. 18A through 18D show further embodiments of medical adhesive applicators. FIG. 18A shows a medical adhesive applicator 200a. The medical adhesive applicator 200a may be similar in function and have many similar components as medical adhesive applicator 200 described above and others described herein. The medical adhesive applicator 200a may comprise the adhesive layer 230. The adhesive layer 230 may comprise a bottom surface removably adhered to the double-layered, adhesive liner 220 which may be pulled away from the bottom surface of the adhesive layer 230 with a proximal tab 225. As described above, pulling the liner 220 away may both absorb excess fluid from the incision I and facilitate adhesion of the bottom surface of the adhesive layer 230 to the tissue T near the incision I. The upper surface 230a of the adhesive layer 230 may be non-adhesive. For example, the upper surface 230a of the adhesive layer 230 may comprise a thin, polyurethane layer. The upper surface of the adhesive layer 230 may be coupled to a distal cuff 240 which has a cavity or opening to receive the distal end of an elongate member 210a. A user can tuck the distal end of the elongate member 210a into the distal cuff 240 to push/insert the adhesive layer 230 and the liner 220 axially into the space 42. Subsequently, the user may remove the elongate member 210a and then the liner 220 to adhere the adhesive layer 230 onto the tissue T over the incision I.

FIG. 18B shows a medical adhesive applicator 200b. The medical adhesive applicator 200b may be similar in function and have many similar components as medical adhesive applicators 200 and 200a described above and other applicators described herein. The applicator 200b may comprise the adhesive layer 230 and a removable, double-layered, absorbent liner 220 having a proximal tab 225. An elongate member 210b has a proximal end which is coupled to the distal tab 235 of the adhesive layer 230. The elongate member 210b may be separated from the upper surface 230a of the adhesive layer 230 by tearing the elongate member 210b away at perforations 201b1 and/or by pulling off a releasable tape laminate 210b2. A user can first axially insert the elongate member 210b into the space 42. Subsequently, the elongate member 210b can be axially pulled to bring the adhesive layer 230 and the liner 220 into the space 42. Then, the user may remove the elongate member 210 and then the liner 220 to adhere the adhesive layer 230 onto the tissue T over the incision I, and the elongate member 210b may be removed from the adhesive layer 230.

FIGS. 18C1 and 18C2 show a medical adhesive applicator 200c. The medical adhesive applicator 200c may be similar in function and have many similar components as medical adhesive applicators 200, 200a, and 200b described above and other applicators described herein. Instead of being inserted axially into the space 42, the medical adhesive applicator 200c may be overlaid over the straps 42 of the incision closure device 10, 100 as shown in FIG. 18C1. As shown in FIG. 18C2, the medical adhesive applicator 200c may be laid over the incision closure device 10, 100 gradually from one lateral side to the next in the direction indicated by arrow 250a. The double-layered absorbent layer 220c may be removed by laterally pulling the layer 220c in the direction indicated by arrow 250b to absorb excess fluid from the incision I and to place the adhesive pads 230c over the tissue T adjacent the incision I. The medical device applicator 200c may further comprise an upper liner layer 230c to cover the adhesive pads 230c. The upper liner layer 230c may have an adhesive bottom surface to hold the adhesive pads 230c in place relative to the incision closure device 10, 100.

FIG. 18D shows a medical adhesive applicator 200d. The medical adhesive applicator 200d may be similar in function and have many similar components as medical adhesive applicators 200, 200a, 200b, and 200c above and other applicators described herein. Instead of being inserted axially into the space 42, the medical adhesive applicator 200d may be overlaid over the straps 42 of the incision closure device 10, 100 as shown in FIG. 18D. The medical adhesive applicator 200d may be axially laid over the incision closure device 10, 100 gradually from one axial end of the incision closure device 10, 100 to the other in the direction indicated by arrow 260a. Once laid on top of the incision closure device 10, 100, the double-layered absorbent layer 220d may be removed axially by pulling the layer 220d in the direction indicated by arrow 260b to absorb excess fluid from the incision I and to place the adhesive pads 230d over the tissue T adjacent the incision I. The medical device applicator 200d may further comprise an upper liner layer 230d to cover the adhesive pads 230d. The upper liner layer 230d may have an adhesive bottom surface to hold the adhesive pads 230d in place relative to the incision closure device 10, 100.

One or more of the components of the incision closure appliances or incision closure appliance assemblies disclosed herein, including one or more of the various base assemblies, base panels, force distribution structures, axial supports, lateral supports, closure components, tie assemblies, straps, locks, adhesive layers, adhesive layers, covers, cover structures, drapes, etc., may be comprised of, be coated with, or otherwise incorporate one or more of an antifungal, antibacterial, antimicrobial, antiseptic, or medicated material. For example, such materials may be incorporated into the hydrocolloid adhesive layer, as another layer or coating between the skin and the adhesive layer (covering at least a portion of the adhesive layer), incorporated into the base assembly cover or at least its adhesive layer, etc. One or more wells, grooves, openings, pores, or similar structures may be provided on the device or apparatus components to facilitate such incorporation. In many embodiments, such materials may comprise one or more of silver, iodide, zinc, chlorine, copper, or natural materials such as tea tree oil as the active agent. Examples of such antifungal, antibacterial, antimicrobial, antiseptic, or medicated materials include, but are not limited to, the Acticoat™ family of materials available from Smith & Nephew plc of the U.K., the Acticoat® Moisture Control family of materials available from Smith & Nephew plc of the U.K., the Contreet® Foam family of materials available from Coloplast A/S of Denmark, the UrgoCell® Silver family of materials available from Urgo Limited of the U.K. (a subsidiary of Laboratoires URGO of France), the Contreet® Hydrocolloid family of materials available from Smith & Nephew plc of the U.K., the Aquacel® Ag family of materials available from ConvaTec Inc. of Skillman, N.J., the Silvercel® family of materials available from Kinetic Concepts, Inc. of San Antonio, Tex., Actisorb® Silver 220 available from Kinetic Concepts, Inc. of San Antonio, Tex., the Urgotul® SSD family of materials available from Urgo Limited of the U.K. (a subsidiary of Laboratoires URGO of France), the Inadine® family of materials available from Kinetic Concepts, Inc. of San Antonio, Tex., the Iodoflex® family of materials available from Smith & Nephew plc of the U.K., the Sorbsan Silver™ family of materials available from Aspen Medical Europe Ltd. of the U.K., the Polymem Silver® family of materials available from Ferris Mfg. Corp. of Burr Ridge, Ill., the Promogram™ family of materials available from Kinetic Concepts, Inc. of San Antonio, Tex., the Promogram Prisma™ family of materials available from Kinetic Concepts, Inc. of San Antonio, Tex., and the Arglaes® family of materials available from Medline Industries, Inc. of Mundelein, Ill. Components of the closure devices described in commonly owned U.S. Pat. Nos. 8,313,508, 8,323,313, and 8,439,945; U.S. Patent Publication No. 2013/0066365; and PCT application nos. US 2010/000430, US 2011/139912, US 2011/40213, US 2011/34649, and US 2013/067024 may also be comprised of, be coated with, or otherwise incorporate one or more of an antifungal, antibacterial, antimicrobial, antiseptic, or medicated material, including but not limited to one or more of the materials listed above.

In many embodiments, topical medicinal agents are incorporated directly into the wound closure appliances described herein. Because a wound closure device is often applied in close proximity to a wound or incision in need of medicinal protection, the incorporation of such medicines directly into the closure device may be beneficial. In wounds at risk of infection, incorporation of anti-microbial agents may be beneficial, for example. Anti-microbial agents may include antibiotic medicines as well as antiseptic metal ions and associated compounds which may include silver, iodine, copper, and chlorine, or natural materials such as tea tree oil. In wounds prone to fungus, medicinal agents such as zinc may be warranted, for example. Combinations of any of these agents may also be of benefit and thus may be incorporated into wound closure appliances.

Topical medicinal agents may be incorporated into the closure devices in a way to give the closure devices the ability to wick exudate away from the wound (e.g., to direct unwanted organisms away from the wound and/or prevent skin maceration), while keeping the wound sufficiently hydrated for improved healing.

While the incision closure appliances 10 and 100 and methods for their use are described herein, many aspects of the present disclosure are applicable for other incision closure appliances described in commonly owned patent applications: U.S. patent applications Ser. No. 13/685,909 [Attorney Docket No. 35383-705.301]; Ser. No. 13/414,176 [Attorney Docket No. 35383-706.301; Ser. No. 13/096,602, now U.S. Pat. No. 8,439,945 [Attorney Docket No. 35383-707.201]; Ser. No. 13/286,378, now U.S. Pat. No. 8,313,508 [Attorney Docket No. 35383-707.301]; Ser. No. 13/874,046 [Attorney Docket No. 35383-707.302]; Ser. No. 13/286,757, now U.S. Pat. No. 8,323,313 [Attorney Docket No. 35383-709.201]; Ser. No. 13/665,160 [Attorney Docket No. 35383-709.501]; Ser. No. 14/180,564 [Attorney Docket No. 35383-709.502]; and, Ser. No. 14/180,524 [Attorney Docket No. 35383-712.201], for example.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the invention and that methods and structures within the scope of these claims and their equivalents be covered thereby.

1. A method of forming an incision in tissue, said method comprising:
adhering right and left base panels of an incision closure apparatus to a surface of the tissue, wherein an incision is formed in the tissue surface between inner edges of the panels, and wherein the incised tissue edges are separated to perform a procedure, the inner edges of the panels being able to stretch and conform along with the tissue edges while an outer edge and lateral extent of each panel remain dimensionally stable;
securing a plurality of closure components to the right and left base panels to draw the inner edges of the panels back together; and
applying an adhesive between the inner edges of the panels between axially adjacent closure components.

2. The method of claim 1, wherein the adhesive is applied with an external applicator.

3. The method of claim 1, wherein applying the adhesive comprises introducing an adhesive applicator between the inner edges of the panels.

4. The method of claim 3, wherein the adhesive applicator, when introduced between the inner edges of the panels, is disposed over the tissue surface and below the plurality of closure components.

5. The method of claim 3, wherein fluid from the incision is absorbed with an absorbent liner of the adhesive applicator.

6. The method of claim 5, further comprising peeling the absorbent liner away from an adhesive layer of the adhesive applicator.

7. The method of claim 6, further comprising adhering the adhesive layer to the tissue surface and removing a stiff member of the adhesive applicator from the adhesive layer.

8. The method of claim 1, wherein the adhesive comprises one or more of a cyanoacrylate, fibrin, albuim, or a glutaraldehyde based adhesive.

9. An apparatus for applying an adhesive to a surface, the apparatus comprising:
an elongate stiff member having a bottom surface;
an adhesive layer removably coupled to the bottom surface of the elongate stiff member, the adhesive layer having an adhesive bottom surface; and
an absorbent liner removably coupled to the adhesive bottom surface of the adhesive layer.

10. The apparatus of claim 9, wherein the elongate stiff member has a distal end and the adhesive layer comprises a distal tab disposed distally of the distal end.

11. The apparatus of claim 9, wherein the elongate stiff member has a proximal end and the absorbent liner comprises a proximal tab disposed proximally of the proximal end.

12. The apparatus of claim 9, wherein the adhesive layer comprises one or more of a cyanoacrylate, fibrin, albuim, or a glutaraldehyde based adhesive.

13. The apparatus of claim 9, wherein the absorbent liner is folded onto itself

14. A kit for forming an incision in tissue, the kit comprising:
an incision closure apparatus comprising:
a base including a left panel and a right panel, each panel having a tissue adherent lower surface, an upper surface, an inner edge, and an outer edge;
a force distribution structure coupled to each panel, wherein each force distribution structure includes a spine disposed axially adjacent the inner or outer edge to limit axial expansion along the edge and a plurality of axially spaced-apart structures extending laterally from the spine toward the inner or outer edge to limit lateral expansion while allowing axial expansion along the edge; and a closure component which releaseably attaches to the force distribution structure to draw the inner edges of the panels together after they are adhered to tissue on opposite sides of an incision; and
an adhesive for application to the tissue.

15. The kit of claim 14, further comprising instructions to apply the adhesive to tissue between the inner edges of the panels after the panels are adhered to tissue on the opposite sides of the incision.

16. The kit of claim 14, wherein the adhesive comprises one or more of a cyanoacrylate, fibrin, albuim, or a glutaraldehyde based adhesive.

17. The kit of claim 14, further comprising an adhesive applicator for applying the adhesive.

18. The kit of claim 17, wherein the adhesive applicator comprises one or more of a spray gun, an applicator tube, an applicator pen, or a brush.

19. The kit of claim 17, wherein the adhesive applicator comprises:
an elongate stiff member having a bottom surface;
an adhesive layer removably coupled to the bottom surface of the elongate stiff member, the adhesive layer having an adhesive bottom surface; and
an absorbent liner removably coupled to the adhesive bottom surface of the adhesive layer.
